(19) United States
(12) Patent Application Publication
(10) Pub. No.: US 2010/0048903 A1
(43) Pub. Date: Feb. 25, 2010

(54) Method For Producing Azoniaspironortropine Esters And Nortropan-3-One Compounds

(76) Inventors: Andreas Krebs, Mannheim (DE); Bernd Schäfer, Dierbach (DE); Arno Kochner, Waldsee (DE)

Correspondence Address: MCANDREWS HELD & MALLOY, LTD, 500 WEST MADISON STREET, SUITE 3400, CHICAGO, IL, 60661, US

(21) Appl. No.: 12/543,193

(22) Filed: Aug. 18, 2009

Related U.S. Application Data
(63) Continuation of application No. PCT/EP2008/001444, filed on Feb. 22, 2008.

(30) Foreign Application Priority Data
Feb. 23, 2007 (EP) EP 07102978.9
Feb. 26, 2007 (EP) EP 07103074.6

(51) Int. Cl. C07D 451/00; C07D 451/02

(52) U.S. Cl. 546/18; 546/127

(57) ABSTRACT

The present technology relates to an improved one-stage method for producing azoniaspironortropine esters such as trospium chloride by reacting an endonortropine compound with an organic dihalogen compound and an a-hydroxycarboxylic acid in the presence of a base and 1,1′ carbonyldiimidazole or 1,1′ thiocarbonyldiimidazole or thionyldiimidazole. The present technology also relates to an improved method for producing nortropan-3-one compounds or their hydrohalides (such as N-benzyltropanone hydrochloride) by reacting an amine and a protected dialdehyde with a basic aqueous solution of 1,3-acetone dicarboxylic acid.

### TECHNICAL FIELD OF THE INVENTION

The present technology relates to an improved single-stage method for producing azoniaspironortropine esters, in particular trospium halides, such as, for example, trospium chloride, by reacting endo-nortropine or a derivative thereof with an organic dihalogen compound, such as 1,4-dihalobutane and an α-hydroxycarboxylic acid such as benzylic acid in the presence of a base and an activating reagent selected from the group 1,1′-carbonyldiimidazole, 1,1′-thiocarbonyldiimidazole and thionyldiimidazole. Furthermore, the present technology relates to an improved method for producing protected nortropan-3-one compounds such as 8-benzylnortropan-3-one and hydrohalides thereof, in particular hydrochlorides, such as N-benzyltropanone hydrochloride, by reacting an amine, such as benzylamine, and a protected dialdehyde with a basic aqueous solution of 1,3-acetonedicarboxylic acid. The nortropan-3-one compounds produced according to the present technology can be converted to endo-nortropine compounds, which in turn can be used as starting materials in the method according to the present technology for producing azoniaspironortropine esters.

### STATE OF THE ART

Trospium chloride is a very potent spasmolytic. To produce this active ingredient, a series of methods has hitherto been described in which trospium chloride is produced starting from the azoniaspiro compound of the formula VII.

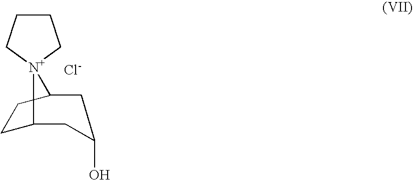

In DE 1194422 and H. Bertholdt, R. Pfleger, W. Schulz, Arzneimittelforschung [Drug Research] (1967), 719, the azoniaspiro compound of the formula VII is reacted with chlorodiphenylacetyl chloride. The process is involved because the active ingredient is obtained only as a result of substitution of the chlorine in the chlorodiphenyl ester. Furthermore, a major disadvantage is the low solubility of compound VII in virtually all aprotic solvents.

According to the teaching of DE 3546218, the compound of the formula VII is reacted with benzylic acid imidazolide of the formula VIII.

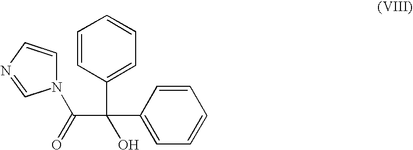

It is a disadvantage of the process that both compounds have to be isolated in pure form before they can be reacted with one another. The concentration of the compound VII at the start of the reaction is below 1% by weight.

8-Benzylnortropan-3-one is a known precursor for producing trospium chloride. To produce this active ingredient, a series of methods has hitherto been described which in all cases start from the feed materials benzylamine, 2,5-dimethoxytetrahydrofuran and acetonedicarboxylic acid, from which, in a first stage, in the course of a reaction known as a Robinson-Schöpf reaction, N-benzyltropanone hydrochloride is obtained.

WO 2005/080389 describes the production of 8-benzylnortropan-3-one by adding acetonedicarboxylic acid in aqueous solution to the hydrochloric solution of an excess of benzylamine and 2,5-dimethoxytetrahydrofuran and subsequently adding sodium hydrogenphosphate. After adding the sodium hydrogenphosphate, a pH of about 4.5 is established by adding 40% strength sodium hydroxide solution. The reaction requires 2 days under the stated conditions in order to obtain a crude product with a yield of 33%.

WO 2005/101989 describes the rapid addition of acetonedicarboxylic acid as solid and benzylamine to a hydrochloric solution of 2,5-dimethoxytetrahydrofuran. A further additive is sodium acetate. The reaction is firstly started at room temperature and is later completed at 50° C. Here, the product is obtained with a yield of 32%.

In Tetrahedron 2001, 57, 235-239, A. Agócs et al. describe the reaction of an asymmetrically functionalized dialdehyde with benzylamine and acetonedicarboxylic acid. Here, benzylamine and acetonedicarboxylic acid is added to the dialdehyde in water with acetic acid and sodium acetate in one portion.

In Synlett 2005, 164-166, G. P. Pollini et al. describe the reaction of methylamine with a dialdehyde and acetonedicarboxylic acid. Here, the dialdehyde is added to an aqueous solution of acetonedicarboxylic acid and methylamine, which solution is buffered through the use of citric acid.

### OBJECT OF THE INVENTION

It was an object of the present technology to provide an improved method for producing azoniaspironortropine esters, such as, for example, trospium chloride, starting from endo-nortropine, which is readily soluble in aprotic solvents, or derivatives thereof, which makes accessible the specified compounds with the lowest possible total number of stages in the highest possible total yield and in a purity acceptable for drugs and pharmaceutical products.

It was also an object of the present technology to provide an improved method for producing endo-nortropine, one of the starting materials of the trospium chloride synthesis, or derivatives thereof via protected nortropan-3-one compounds as intermediates. During the production of protected nortropan-3-one compounds, two moles of carbon dioxide are formed during the reaction as a result of the decarboxylation of the carboxylic acid functionalities introduced via the 1,3-acetonedicarboxylic acid used per mole of 1,3-acetonedicarboxylic acid used. For reactions on an industrial scale, the release of large amounts of a gaseous by-product constitutes a challenge from an apparatus and safety point of view. In this connection, as controlled as possible a gas release is desired. Moreover, the aim was to find conditions under which the production of protected nortropan-3-one compounds, such as 8-benzylnortropan-3-one hydrochloride, can be carried out by a Robinson-Schöpf reaction in the highest possible yield and with a small amount of by-products and/or impurities which can be separated off as easily as possible.

### DESCRIPTION OF THE INVENTION AND OF THE PREFERRED EMBODIMENTS

In a first aspect, the present technology relates to a method for producing azoniaspironortropine ester halide compounds of the formula I

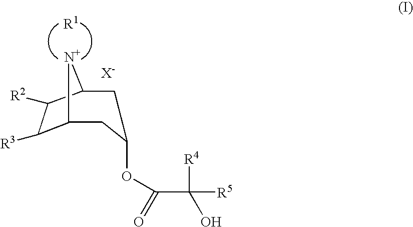

where
X is a halogen atom,
R¹ is selected from the group consisting of optionally substituted alkylene group, optionally substituted alkenylene group and the group —R—Z—R—, where each R, independently of the others, is a covalent bond, an optionally substituted alkylene group or an optionally substituted alkenylene group, where both R groups may not be a covalent bond at the same time, and Z is an optionally substituted cycloalkyl group, an optionally substituted heterocyclyl group, an optionally substituted aryl group or an optionally substituted heteroaryl group,
R² and R³, independently of one another, are selected from the group consisting of hydrogen, optionally substituted alkyl group and optionally substituted cycloalkyl group, and
R⁴ and R⁵, independently of one another, are selected from the group consisting of hydrogen, halogen atom, optionally substituted alkyl group, optionally substituted alkenyl group, optionally substituted cycloalkyl group, optionally substituted heterocyclyl group, optionally substituted aryl group and optionally substituted heteroaryl group,
by reacting a compound of the formula II

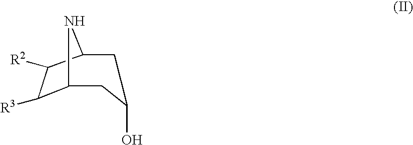

where
R² and R³ each have the same meaning as in formula I, with a compound of the formula III

X—R¹—X¹ (III)

where
X and R¹ each have the same meaning as in formula I, and
X¹ is a halogen atom,
and with a compound of the formula IV

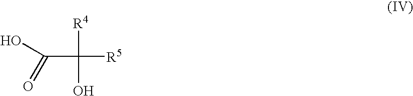

where
R⁴ and R⁵ each have the same meaning as in formula I,
in the presence of a base of the formula V

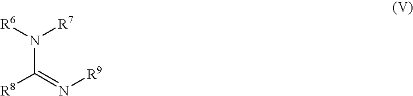

where
R⁶, R⁷ and R⁹ may be identical or different and, independently of one another, are hydrogen or an optionally substituted, saturated or unsaturated hydrocarbon radical, where R⁷ and R⁹ may together also form an optionally substituted, saturated or unsaturated hydrocarbon bridge, and
R⁸ is hydrogen or an optionally substituted, saturated or unsaturated hydrocarbon radical or the radical —NR¹⁰R¹¹, where the radicals R¹⁰ and R¹¹ may be identical or different and are hydrogen or an optionally substituted, saturated or unsaturated hydrocarbon radical, where the radicals R⁶ and R⁸ may together also form an optionally substituted hydrocarbon bridge,
and in the presence of at least one activating reagent selected from the group of activating reagents 1,1′-carbonyldiimidazole, 1,1′-thiocarbonyldiimidazole and thionyldiimidazole.

In a preferred embodiment, the present technology relates to a method for producing trospium halide of the formula Ia

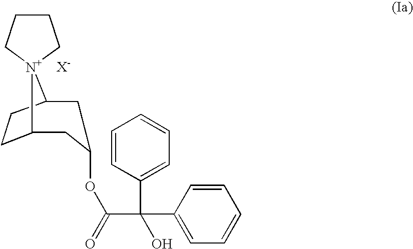

where
X is chlorine, bromine or iodine,
by reacting endo-nortropine of the formula IIa

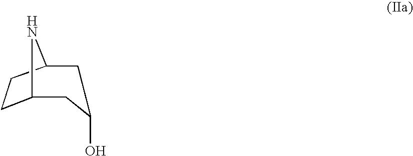

with 1,4-dihalobutane of the formula IIIa

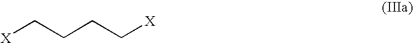

where
X has the same meaning as in formula Ia
and with benzylic acid of the formula IVa

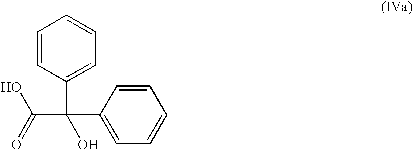

in the presence of a base of the formula Va

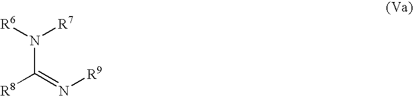

in which the radicals
R⁶, R⁷ and R⁹ may be identical or different and, independently of one another, are hydrogen or a saturated or unsaturated hydrocarbon radical having 1 to 4 carbon atoms, where R⁷ and R⁹ may together also form a saturated or unsaturated hydrocarbon bridge having 3 to 6 carbon atoms, and
R⁸ is hydrogen or a saturated or unsaturated hydrocarbon radical having 1 to 4 carbon atoms or the radical —NR¹⁰R¹¹, where the radicals R¹⁰ and R¹¹ may be identical or different and are hydrogen or a saturated or unsaturated hydrocarbon radical having 1 to 4 carbon atoms, and where the radicals R⁶ and R⁸ may together also form a hydrocarbon bridge having 3 to 6 carbon atoms,
and in the presence of at least one activating reagent selected from the group of activating reagents 1,1′-carbonyldiimidazole, 1,1′-thiocarbonyldiimidazole and thionyldiimidazole.

Through the method according to the present technology, azoniaspironortropine ester halide compounds such as trospium halides of the formula Ia

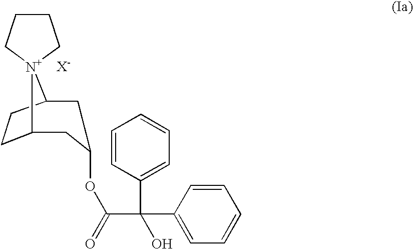

are accessible, where X is preferably a chlorine, bromine or iodine atom, more preferably a chlorine or bromine atom and particularly preferably a chlorine atom. A process product preferred according to the present technology is therefore trospium chloride.

Suitable starting compounds for carrying out the method according to the present technology are endo-nortropine compounds of the formula II, preferably endo-nortropine (8-azabicyclo[3.2.1]octan-3-ol), and α-hydroxycarboxylic acids of the formula IV, preferably benzylic acid (hydroxydiphenylacetic acid), and organic dihalogen compounds of the formula III, preferably 1,4-dihalobutane of the formula IIIa

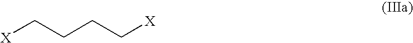

where X in each case has the same meaning as in the process product of formula I and is preferably a chlorine, bromine or iodine atom, more preferably a chlorine or bromine atom and particularly preferably a chlorine atom.

The reaction according to the present technology is carried out in the presence of a base of the formula V

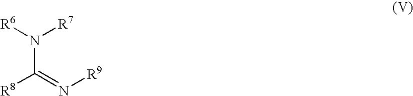

R⁶, R⁷ and R⁹ may be identical or different and are preferably, independently of one another, hydrogen or a saturated or unsaturated hydrocarbon radical having 1 to 4 carbon atoms, where R⁷ and R⁹ may together also form a saturated or unsaturated hydrocarbon bridge having 3 to 6 carbon atoms, and
R⁸ is preferably hydrogen or a saturated or unsaturated hydrocarbon radical having 1 to 4 carbon atoms or the radical —NR¹⁰R¹¹, where the radicals R¹⁰ and R¹¹ may be identical or different and are hydrogen or a saturated or unsaturated hydrocarbon radical having 1 to 4 carbon atoms, and where the radicals R⁶ and R⁸ may together also form a hydrocarbon bridge having 3 to 6 carbon atoms.

Among the possible amidines, guanidines or imidazoles of the formula V, bases preferred according to the present technology are: imidazole, 1,8-diazabicyclo[4.3.0]non-5-ene (DBN) and 1,8-diazabicyclo[5.4.0]undec-7-ene (DBU), particularly preferably imidazole.

Moreover, the reaction according to the present technology is carried out in the presence, i.e. with use, of an activating reagent selected from the group of activating reagents 1,1′-carbonyldiimidazole, 1,1′-thiocarbonyldiimidazole and thionyldiimidazole. Here, the activating reagents 1,1′-carbonyldiimidazole and 1,1′-thiocarbonyldiimidazole are described by formula VI

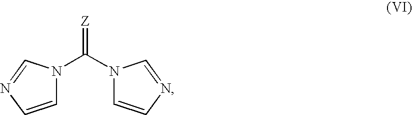

where, in the case of 1,1′-carbonyldiimidazole, Z is oxygen, and in the case of 1,1′-thiocarbonyldiimidazole, Z is sulfur. Thionyldiimidazole has the formula IX.

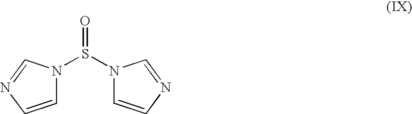

Within the context of a preferred embodiment, the method according to the present technology is carried out in the presence of 1,1′-carbonyldiimidazole (CDI) or 1,1′-thiocarbonyldiimidazole, particularly preferably in the presence of 1,1′-carbonyldiimidazole.

In preferred embodiments of the present technology, X and X¹, independently of one another, are a chlorine, bromine or iodine atom, preferably a chlorine or bromine atom and particularly preferably a chlorine atom. In one embodiment, X and X¹ are the same halogen atom. The ring

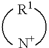

in the compound of the formula I has preferably at least 4, more preferably at least 5, ring atoms and preferably up to 10, more preferably up to 8, more preferably up to 6, ring atoms. R¹ is preferably an optionally substituted alkylene group or an optionally substituted alkenylene group, more preferably an alkylene group having 3 to 9 carbon atoms, preferably having 3 to 7 carbon atoms and more preferably having 4 or 5 carbon atoms. Preferably, R¹ is a 1,4-butylene group. R² and R³, independently of one another, are preferably hydrogen or an optionally substituted alkyl group, more preferably hydrogen or an alkyl group having 1 to 4 carbon atoms and particularly preferably hydrogen. R⁴ and R⁵, independently of one another, are preferably selected from the group consisting of optionally substituted alkyl group, optionally substituted alkenyl group, optionally substituted cycloalkyl group and optionally substituted aryl group. More preferably, R⁴ and R⁵ are optionally substituted aryl groups and particularly preferably phenyl groups.

The specified starting materials and reagents can be used in standard commercial grades and purities of from about 90 to 100% by weight and generally require no special purification prior to use. Thionyldiimidazole is produced by methods known to the person skilled in the art e.g. from thionyl chloride and imidazole (Takahashi, Shimizu, Ogata, Heterocycles (1985) 1483).

The reaction according to the present technology is advantageously carried out in an organic solvent or a mixture of different organic solvents. Of particular suitability are aprotic polar organic solvents such as, for example, N,N-dimethylformamide (DMF), dimethylacetamide and N-methylpyrrolidone or mixtures thereof. The specified solvents can comprise small amounts of water and do not have to be used in a specially predried form. According to one preferred embodiment, the reaction according to the present technology is carried out in dimethylformamide as solvent. The specified solvents, preferably DMF, are preferably used in an amount such that the resulting reaction mixtures have a concentration of endo-nortropine compound of the formula II, such as, for example, endo-nortropine, of from about 5 to about 30% by weight, preferably about 5 to about 10% by weight (based on the reaction mixture).

The quantitative ratio of the starting materials to be used according to the present technology can be varied within wide limits. Taking into consideration the cost aspect, the endo-nortropine compound of the formula II, e.g. endo-nortropine, and the α-hydroxycarboxylic acid of the formula IV, e.g. benzylic acid, are used in a molar ratio of from about 1:3 to about 3:1, preferably in a molar ratio of from about 1:1 to about 1:2. The molar ratio of the endo-nortropine compound of the formula II used, e.g. endo-nortropine, to the organic dihalogen compound of the formula III, e.g. 1,4-dihalobutane, is also preferably selected in a range from about 3:1 to about 1:3, preferably from about 1:1 to about 1:2.

The order in which the specified starting materials and reagents are added to the reaction mixture is generally not critical. Usually, the endo-nortropine compound of the formula II, e.g. endo-nortropine, is initially introduced with the organic dihalogen compound of the formula III, e.g. 1,4-dihalobutane, in the presence of a base, heated for about 1 to about 8 h at temperatures of about 60 to about 100° C. and then this mixture is admixed at about 10 to about 100° C. with the α-hydroxycarboxylic acid of the formula IV, e.g. benzylic acid, and the selected activating reagent of the formula VI (1,1′-carbonyldiimidazole or 1,1′-thiocarbonyldiimidazole) or IX (thionyldiimidazole).

Alternatively, it is also possible to add a mixture formed from the activating reagent of the formula VI or IX and the α-hydroxycarboxylic acid of the formula IV, e.g. benzylic acid (i.e. the activated α-hydroxy-carboxylic acid) to the remaining components, which have been treated beforehand as described above. Alternatively, the reaction mixture, treated as described above, of the endo-nortropine compound of the formula II, e.g. endo-nortropine, the organic dihalogen compound of the formula III, e.g. 1,4-dihalobutane, and the base of the formula V can also be admixed with the α-hydroxycarboxylic acid of the formula IV, e.g. benzylic acid, and finally the activating reagent of the formula VI and/or IX can be added at about 10 to about 100° C.

Surprisingly, it is furthermore possible to react the endo-nortropine compound of the formula II, e.g. endo-nortropine, with the α-hydroxycarboxylic acid of the formula IV, e.g. benzylic acid, and the activating reagent of the formula VI or IX at about 10 to about 100° C., then to add the organic dihalogen compound of the formula III, e.g. 1,4-dihalobutane, and the base of the formula V and to heat for about 1 to about 8 h at temperatures of about 60 to about 100° C.

Within the context of an embodiment preferred according to the present technology, the method is carried out in the presence of 1,1′-carbonyldiimidazole or 1,1′-thiocarbonyldiimidazole or thionyldiimidazole without the separate addition of a base of the formula V. The base of the formula V used in these cases is the imidazole released in situ by the reaction of 1,1′-carbonyldiimidazole or 1,1′-thiocarbonyldiimidazole or thionyldiimidazole with the α-hydroxycarboxylic acid of the formula IV used, e.g. benzylic acid, which renders the separate addition of imidazole or of a further base superfluous. In this case, preferably the endo-nortropine compound of the formula II, e.g. endo-nortropine, is reacted with the α-hydroxycarboxylic acid of the formula IV, e.g. benzylic acid, and the selected activating reagent of the formula VI or IX at about 10 to about 40° C., the organic dihalogen compound of the formula III, e.g. 1,4-dihalobutane, is added and the mixture is heated for about 1 to about 8 h at temperatures of about 60 to about 100° C.

The specified starting materials and reagents can also be introduced simultaneously into a suitable reaction vessel, e.g. a flow reactor or a battery of tank reactors, in the sense of a continuous procedure. In each case, it is advantageous to ensure effective mixing of the specified reaction components, for example by using a suitable stirrer.

To successfully carry out the production method according to the present technology, the use of a catalyst is not absolutely necessary, although it may be an advantageous influence. In particular, the use of nucleophilic catalysts has proven advantageous. A preferred catalyst according to the present technology in this regard is 4-(N,N-dimethylamino)pyridine (DMAP). A preferred embodiment of the method according to the present technology is accordingly characterized in that it is carried out in the presence of 4-(N,N-dimethylamino)pyridine as catalyst.

The reaction according to the present technology can be carried out under atmospheric pressure. If desired, it is also possible to work at a pressure of up to 200 bar.

It may be advisable to carry out the reaction under an inert protective gas atmosphere, it being possible to use nitrogen, carbon dioxide or argon as protective gases.

The reaction time is generally governed by the reaction temperature at which the components used, i.e. starting materials and reagents, are reacted with one another. The end of the reaction can be established using customary methods, for example capillary zone electrophoresis, ion exchange chromatography, thin-film chromatography or HPLC.

The method according to the present technology opens up an operationally easy and efficient access to azoniaspironortropine ester halide compounds of the formula I such as, for example, trospium halides of the formula Ia. Here, the fact that the process products according to the present technology can be obtained by a single-stage reaction, i.e. without isolation or purification of intermediates, is operationally and economically particularly advantageous. As a result, complex work-up, purification and isolation measures are saved and a higher yield is achieved. Accordingly, a single-stage process procedure in which no intermediates are isolated is a preferred embodiment of the method according to the present technology.

The method according to the present technology opens up, for example, access to either trospium chloride, bromide or iodide depending on which 1,4-halobutane of the formula IIIa is used. Using 1,4-dibromobutane or 1,4-diiodobutane accordingly gives trospium bromide or trospium iodide, respectively, which, if desired, can in each case be converted to trospium chloride through treatment with suitable ion exchangers in accordance with the teaching of DE 119 44 22. As regards the production of trospium chloride, the option of using 1,4-dichlorobutane, which is more cost-effective and more advantageous from a safety aspect, constitutes a further advantage of the method according to the present technology.

After the reaction has taken place, the resulting azoniaspironortropine ester halide compound such as trospium halide, preferably trospium chloride, can be isolated from the reaction mixture by customary methods per se, for example by extraction and crystallization, and/or be further purified. Base/solvent pairs suitable for this purpose are in particular those in which the corresponding undesired hydrohalides remain dissolved in the solvent at the filtration temperature, but the azoniaspironortropine ester halide compound, such as, for example, the trospium halide, precipitates out. In one preferred embodiment of the method according to the present technology, accordingly, the resulting azoniaspironortropine ester halide compound of the formula I, e.g. trospium halide of the formula Ia, is treated with a solvent, for example with N,N-dimethylformamide, in which the azoniaspironortropine ester halide compound, such as, for example, the trospium halide, precipitates out and the hydrohalide of the base of the formula V used remains dissolved.

For this, it may be necessary to use the selected solvent in an amount such that the base of the formula V used, preferably imidazole or diazabicyclo[5.4.0]undec-7-ene, precipitate out not in the form of their hydrohalides, specifically not in the form of their hydrochlorides, but remain dissolved and can thus be easily separated from the azoniaspironortropine ester halide compound such as trospium halide. Depending on the type of solvent selected for the purification and the amount used, i.e. depending on the resulting concentration of the product and/or the by-products to be separated off, it may be advantageous to select the temperature so that the desired process product precipitates out and the undesired by-products remain dissolved. A preferred example of this is 1,8-diazabicyclo[5.4.0]undec-7-ene hydrochloride in DMF at about 0° C.

In a second aspect, the present technology relates to a method for producing N-substituted nortropan-3-one compounds. These compounds can be converted to endo-nortropine compounds of the formula II, which serve as starting materials in the method according to the present technology for producing azoniaspironortropine ester halide compounds of the formula I. For example, through a first reaction, the protective group of the amine can be cleaved off and, through a further reaction, the keto group can be converted to a hydroxyl group. Preferably, the elimination of the protective group, which is preferably a benzyl group, takes place by means of hydrogenolysis, for example with hydrogen and a Pd/C catalyst in an acidic aqueous solvent such as a hydrochloric isopropanol/water mixture. The reduction of the keto group preferably takes place by means of hydrogen and a Raney nickel catalyst in a basic aqueous solvent such as an isopropanol/water mixture with sodium hydroxide and subsequent treatment with toluene. The resulting product is then preferably isolated as free base by means of azeotropic distillation from a mixture of an organic and an aqueous solvent such as toluene/water and crystallization.

In a second aspect, the present technology therefore relates to a method for producing nortropan-3-one compounds of the formula X

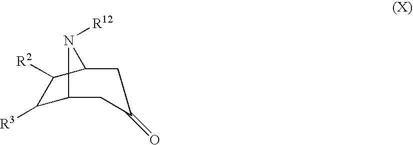

where
R² and R³, independently of one another, are selected from the group consisting of hydrogen, optionally substituted alkyl group and optionally substituted cycloalkyl group, and
R¹² is selected from the group consisting of optionally substituted alkyl group, acyl group and carboxyl ester group,
by reacting a compound of the formula XI

H₂N—R¹² (XI)

where
R¹² has the same meaning as in formula X,
with a compound of the formula XII

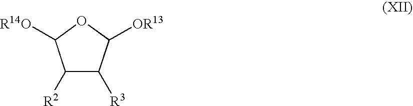

where
R² and R³ each have the same meaning as in formula X, and R¹³ and R¹⁴, independently of one another, are selected from the group consisting of hydrogen, optionally substituted alkyl group, optionally substituted alkenyl group, optionally substituted cycloalkyl group and optionally substituted aryl group,
and 1,3-acetonedicarboxylic acid of the formula XIII

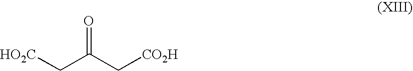

characterized in that 1,3-acetonedicarboxylic acid of the formula XIII is used in the form of an aqueous solution with a pH of greater than 7.

In a preferred embodiment, the present technology relates to a method for producing 8-benzylnortropan-3-one (8-benzyl-8-azabicyclo[3.2.1]octan-3-one) of the formula Xa

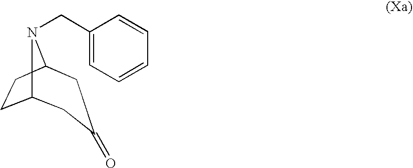

by reacting benzylamine of the formula XIa

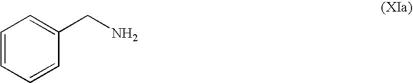

with a compound of the formula XIIa

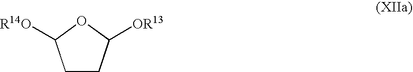

where the radicals
R¹³ and R¹⁴ are identical or different and are in each case hydrogen or an unbranched or branched C₁- to C₆-alkyl group,
and 1,3-acetonedicarboxylic acid of the formula XIII

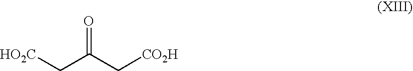

which is characterized in that 1,3-acetonedicarboxylic acid of the formula XIII is used in the form of an aqueous solution with a pH of greater than about 7.

Preferably, R² and R³, independently of one another, are hydrogen or an optionally substituted alkyl group, more preferably hydrogen or an alkyl group having 1 to 4 carbon atoms and particularly preferably hydrogen. R¹² is preferably a protective group which can be cleaved off from the nitrogen through reduction or through basic or acidic treatment, preferably through hydrogenolysis. Examples of such protective groups are a benzyl group, a tert-butoxycarbonyl group, a benzyloxycarbonyl group, a 9-fluorenylmethoxycarbonyl group and an acetyl group. Preferably, R¹² is a benzyl group. The nortropan-3-one compound of the formula X is preferably 8-benzylnortropan-3-one (8-benzyl-8-azabicyclo[3.2.1]octan-3-one).

Within the context of the method according to the present technology, the 1,3-acetonecarboxylic acid is used in the form of an aqueous solution with a pH greater than 7, i.e. in completely or partly, preferably in completely dissolved form in water or a mixture of water and at least partly water-miscible organic solvents, such as, for example, methanol or ethanol and a water-soluble base.

Bases suitable according to the present technology for this purpose are, for example, alkali metal or alkaline earth metal hydroxides, such as, for example, sodium hydroxide, lithium hydroxide, potassium hydroxide, barium hydroxide or calcium hydroxide, preferably sodium hydroxide, lithium hydroxide or potassium hydroxide, particularly preferably sodium hydroxide. The specified bases can be used in pure form or in the form of mixtures with one another. Here, the selected base is used in an amount which suffices to deprotonate the two carboxylic acid groups of the 1,3-acetonedicarboxylic acid used and, moreover, to bring the pH of the aqueous solution of the 1,3-acetonedicarboxylic acid to a value greater than 7, i.e. to an alkaline pH. According to the present technology, preference is given to using 1,3-acetonedicarboxylic acid in dissolved form in aqueous sodium hydroxide solution, lithium hydroxide solution or potassium hydroxide solution, particularly preferably sodium hydroxide solution or potassium hydroxide solution, usually with an alkali metal hydroxide concentration of from about 3 mol/l to about 7 mol/l, preferably about 4 mol/l to about 6 mol/l. The resulting aqueous solutions of 1,3-acetonedicarboxylic acid or the corresponding dicarboxylate usually have a pH of from about 7 to about 14, preferably about 10 to about 14.

The 1,3-acetonedicarboxylic acid to be reacted according to the present technology is present in the aqueous solution with a pH greater than about 7 in completely deprotonated form, i.e. in the form of its dicarboxylate dianion. The concentration of the 1,3-acetonedicarboxylic acid to be reacted or its dianion in the specified alkaline solutions is not critical and can be varied over a wide range, but is usually about 1 to about 5, preferably about 2 to about 3 mol/l.

Suitable further starting materials for carrying out the method according to the present technology are an amine of the formula XI, preferably benzylamine, and a compound of the formula XII, preferably a compound of the formula XIIa

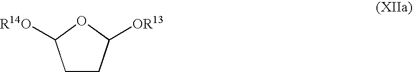

where the radicals R¹³ and R¹⁴ are identical or different, preferably identical and are in each case hydrogen or an unbranched or branched C₁- to C₆-alkyl group. In this connection, straight-chain or branched C₁- to C₆-alkyl group is to be understood as meaning straight-chain or branched alkyl radicals having 1 to 6 carbon atoms, such as, for example, a methyl, ethyl, propyl, isopropyl, butyl, isobutyl, sec-butyl, tert-butyl, pentyl or hexyl group, preferably a methyl, ethyl, propyl or isopropyl group, particularly preferably a methyl or ethyl group and very particularly preferably a methyl group. A compound of the formula XII particularly preferred according to the present technology is accordingly 2,5-dimethoxytetrahydrofuran of the formula XIIb.

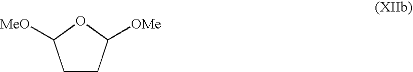

In a manner according to the present technology it is also possible to use compounds of the formula XII, in particular compounds of the formula XIIa, in which one of the radicals R¹³ and R¹⁴ or both radicals are hydrogen.

The specified compounds of the formulae XI and XII, in particular compounds of the formulae XIa and XIIa, can in each case be used as such or in the form of solutions, preferably in the form of aqueous solutions. Possible solvents which may also be mentioned for this purpose are water or mixtures of water and at least partially water-miscible solvents as described above.

Within the context of a preferred embodiment of the method according to the present technology, the amine of the formula XI, preferably benzylamine, and the compound of the formula XII, preferably the compound of the formula XIIa, more preferably 2,5-dimethoxytetrahydrofuran, are used in the form of an aqueous solution with a pH less than about 7. Solvents which can be used for producing such solutions are aqueous solutions of organic or inorganic acids, in particular aqueous hydrochloric acid or phosphoric acid or aqueous solutions of organic carboxylic acids such as, for example, acetic acid. According to a preferred embodiment, the amine of the formula XI, preferably benzylamine, and the compound of the formula XII, preferably the compound of the formula XIIa, more preferably 2,5-dimethoxytetrahydrofuran, are used in the form of an aqueous solution comprising hydrochloric acid. Particular preference is given to using the specified starting materials in the form of a solution in hydrochloric acid.

When using the amine of the formula XI, preferably benzylamine, and the compound of the formula XII, preferably the compound of the formula XIIa, more preferably 2,5-dimethoxytetrahydrofuran, in the form of an aqueous solution with a pH less than about 7, i.e. in the form of an acidic solution, the amine of the formula XI, such as benzylamine, is usually present in protonated form, for example in hydrochloric aqueous solution in the form of its hydrochloride.

The specified starting materials of the method according to the present technology can be used according to the underlying stoichiometry of the reaction in approximately equimolar ratio, it also being possible, if appropriate, to use one or two reaction components in slight deficit or excess. Preferably, the starting materials 1,3-acetonedicarboxylic acid of the formula XIII to the amine of the formula XI, preferably benzylamine, and the compound of the formula XII, preferably the compound of the formula XIIa, more preferably 2,5-dimethoxytetrahydrofuran, are used in a molar ratio of from about 1:1:1 to about 1:0.6:1.2, preferably up to about 1:0.7:1.1.

To carry out the reaction according to the present technology, the 1,3-acetonedicarboxylic acid to be used in the form of an aqueous solution with a pH greater than about 7 is brought into contact with the two other starting materials, the amine of the formula XI, preferably benzylamine, and the compound of the formula XII, preferably the compound of the formula XIIa, more preferably 2,5-dimethoxytetrahydrofuran, as a result of which a reaction mixture is obtained.

The pH of the reaction mixture obtained here can generally vary within a wide range. The reaction is usually carried out under acidic conditions, i.e. at a pH of the reaction mixture of below about 7, preferably at a pH of the reaction mixture of from about 1 to about 6, particularly preferably at a pH of from about 2 to about 5 and particularly preferably at a pH of from about 3 to about 4. When using 1,3-acetonedicarboxylic acid in the form of an aqueous solution with a pH greater than 7, this is achieved, for example, through use of the amine of the formula XI, preferably benzylamine, and the compound of the formula XII, preferably the compound of the formula XIIa, more preferably 2,5-dimethoxytetrahydrofuran, in the form of an aqueous solution with a pH less than about 7, i.e. in the form of an acidic solution.

The reaction according to the present technology is usually carried out at temperatures of from about 0° C. to about 100° C., preferably from about 10° C. to about 40° C. The reaction can be carried out under atmospheric pressure. If desired, it is also possible to work at a pressure of up to 200 bar.

The reaction according to the present technology can be advantageously influenced through suitable selection of reaction conditions. In this connection, it may be in particular advantageous to bring the reactants and starting materials into contact with one another or to combine them in a suitable manner. The reaction can be carried out either continuously or discontinuously, i.e. the starting materials can be brought into contact with one another or combined all at once or be brought together gradually, i.e. spread over a relatively long period.

Thus, for example, the aqueous solution of 1,3-acetonedicarboxylic acid to be used according to the present technology can be initially introduced in a suitable reaction vessel, for example a stirred tank, and the other reactants, the amine of the formula XI, preferably benzylamine, and the compound of the formula XII, preferably the compound of the formula XIIa, more preferably 2,5-dimethoxytetrahydrofuran, can be added in dissolved form or be metered in spread over a relatively long period. Alternatively, the amine of the formula XI, preferably benzylamine, and the compound of the formula XII, preferably the compound of the formula XIIa, more preferably 2,5-dimethoxytetrahydrofuran, can also be initially introduced in solid or dissolved form in a suitable reaction vessel, and the aqueous solution of 1,3-acetonedicarboxylic acid with a pH greater than 7 to be used according to the present technology can be added or metered in. Within the context of a further embodiment of the method according to the present technology, the aqueous solution of 1,3-acetonedicarboxylic acid with a pH greater than about 7 and the other reactants are introduced in parallel, preferably spread over a relatively long period, into a suitable reaction vessel. In this connection, it is in every case advantageous to ensure thorough mixing of the reactants, for example by using a stirrer.

A preferred embodiment of the method according to the present technology is characterized in that an aqueous solution of the amine of the formula XI, preferably benzylamine, and the compound of the formula XII, preferably the compound of the formula XIIa, more preferably 2,5-dimethoxytetrahydrofuran, and the aqueous solution of 1,3-acetonedicarboxylic acid with a pH greater than about 7 are brought into contact with one another so that the aqueous solution of the amine of the formula XI, preferably benzylamine, and the compound of the formula XII, preferably the compound of the formula XIIa, more preferably 2,5-dimethoxytetrahydrofuran, are initially introduced in a reaction vessel and the aqueous solution of 1,3-acetonedicarboxylic acid of the formula XIII is metered in to give a reaction mixture. Preference is given here to using a hydrochloric aqueous solution of the amine of the formula XI, preferably benzylamine, and the compound of the formula XII, preferably the compound of the formula XIIa, more preferably 2,5-dimethoxytetrahydrofuran.

A further preferred embodiment of the method according to the present technology is characterized in that an aqueous solution of the amine of the formula XI, preferably benzylamine, and the compound of the formula XII, preferably the compound of the formula XIIa, more preferably 2,5-dimethoxytetrahydrofuran, and the aqueous solution of 1,3-acetonedicarboxylic acid of the formula XIII are brought into contact with one another such that they are simultaneously metered in to a reaction vessel to give a reaction mixture. Here, the starting materials are brought into contact with one another continuously or else in portions spread over a relatively long period. It is of particular advantage here to use an aqueous solution of the amine of the formula XI, preferably benzylamine, and the compound of the formula XII, preferably the compound of the formula XIIa, more preferably 2,5-dimethoxytetrahydrofuran, with a pH of less than about 7. Preference is given to using a hydrochloric aqueous solution of the specified starting materials.

Through suitable selection of the reaction conditions, it is possible to arrange for the release of relatively large amounts of gaseous carbon dioxide, which occurs in the course of the reaction according to the present technology, in a manner which is technically easy to handle. If, for example, the 1,3-acetonedicarboxylic acid to be reacted is introduced continuously or in portions into the initially charged total amount of the other reactants spread over a relatively long period, the release rate of the gaseous by-product can be controlled. The same applies for the simultaneous addition of the starting materials if the reaction conditions are selected such that the reactants which are added simultaneously react directly at least for the large part and thus results in a continuous release of the gaseous by-product carbon dioxide. This is possible particularly if the reaction conditions, in particular the pH of the aqueous solutions used, are selected so that the reaction mixture which is obtained by bringing the starting materials into contact, has a pH of from about 1 to about 6, particularly preferably from about 2 to about 5 and especially preferably from about 3 to about 4.

Advantageously, a solution of 1,3-acetonedicarboxylic acid in sodium hydroxide solution can be metered in parallel to the amine of the formula XI, preferably benzylamine, and the compound of the formula XII, preferably the compound of the formula XIIa, more preferably 2,5-dimethoxytetrahydrofuran, in hydrochloric acid such that a pH of about 3.5 is kept constant during the reaction. As soon as the addition of the acidic solution has taken place, the remainder of 1,3-acetonedicarboxylic acid solution can be added.

The reaction time is usually governed by the amount of components used and the maximum possible gas removal. The end of the reaction can be established using customary methods, for example by means of thin-film chromatography or HPLC.

After the reaction has taken place, a basic solution, e.g. sodium hydroxide solution, potassium hydroxide solution, preferably sodium hydroxide solution, is advantageously added to the reaction solution to give a mixture comprising the process product according to the present technology of the formula X, preferably formula Xa, which mixture has a pH in the range from about 7 to about 14, preferably in the range from about 9 to about 11.

For the isolation, the nortropan-3-one compound obtained according to the present technology of the formula X, for example 8-benzylnortropan-3-one, can be isolated from the reaction mixture by methods customary per se, for example by extraction. Of suitability for the extraction are aromatic and saturated and unsaturated aliphatic, branched and unbranched hydrocarbons having about 5 to about 12 carbon atoms, in particular toluene, pentane, hexane, heptane and ethyl acetate, very particularly ethyl acetate. Also suitable are aliphatic ethers having about 4 to about 8 carbon atoms, for example diethyl ether or methyl tert-butyl ether and supercritical solvents, such as, for example, carbon dioxide, propane or butane. The amount of solvent added for the extraction, preferably the ethyl acetate added, is generally not critical. Usually, the volume ratio of organic solvent to water is about 0.1:1 to about 1:1, preferably about 0.25:1.

The crude product obtained by extraction in organic phase can then be dried by methods known per se to the person skilled in the art. An azeotropic drying of the organic phase is advantageously carried out in the presence of an alcohol or ester as cosolvent, preferably methanol, ethanol, propanol, isopropanol, butanol, in particular ethyl acetate. The compound of the formula X obtained in the form of the free amine can then be converted to its corresponding acid addition salt through treatment with an acid.

Preferably, the nortropan-3-one compound of the formula X obtained according to the present technology, for example 8-benzylnortropan-3-one, is treated with a hydrohalic acid, preferably hydrochloric acid, e.g. in the form of an aqueous solution such as aqueous hydrochloric acid or an alcoholic solution of hydrohalic acid, preferably of an isopropanolic solution such as isopropanolic HCl, and it is isolated in the form of a nortropan-3-one hydrohalide compound, for example in the form of 8-benzylnortropan-3-one hydrochloride of the formula XIVa.

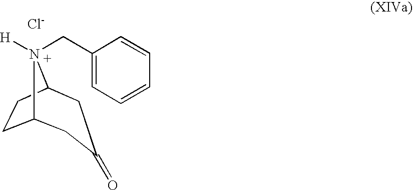

Accordingly, the present technology also relates to a method for producing nortropan-3-one hydrohalide compounds of the formula XIV

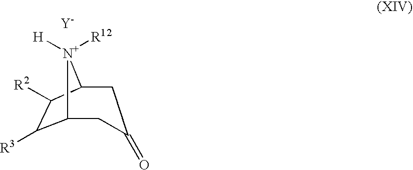

where
R² and R³, independently of one another, are selected from the group consisting of hydrogen, optionally substituted alkyl group and optionally substituted cycloalkyl group,
R¹² is selected from the group consisting of optionally substituted alkyl group, acyl group and carboxyl ester group, and
Y is a halogen atom,
by producing a nortropan-3-one compound according to the method described above and then treating the nortropan-3-one compound produced in this way with a hydrohalic acid.

In one preferred embodiment, the present technology relates to a method for producing 8-benzylnortropan-3-one hydrochloride by producing 8-benzylnortropan-3-one according to the method described above and then treating the 8-benzylnortropan-3-one prepared in this way with hydrochloric acid.

Preferably, R² and R³, independently of one another, are hydrogen or an optionally substituted alkyl group, more preferably hydrogen or an alkyl group having 1 to 4 carbon atoms and particularly preferably hydrogen. R¹² is preferably a protective group which can be cleaved off from the nitrogen by reduction or by basic or acidic treatment, preferably by hydrogenolysis. Examples of such protective groups are a benzyl group, a tert-butoxycarbonyl group, a benzyloxycarbonyl group, a 9-fluorenylmethoxycarbonyl group and an acetyl group. R¹² is preferably a benzyl group. The nortropan-3-one compound of the formula X is preferably 8-benzylnortropan-3-one.

The hydrohalic acid is preferably hydrochloric, hydrobromic or hydroiodic acid, particularly preferably hydrochloric acid, and the nortropan-3-one hydrohalide compound of the formula XIV is correspondingly preferably a nortropan-3-one hydrochloride, nortropan-3-one hydrobromide or nortropan-3-one hydroiodide compound, particularly preferably a nortropan-3-one hydrochloride compound.

For the further purification to be carried out if desired, the solid of the formula XIV, preferably of the formula XIVa, can be suspended in an alcohol, for example in methanol, and be stirred. The substance of value can then be separated by suitable separation methods, for example by filtration, from by-products dissolved in the added methanol which may be present. After another solvent exchange, preferably for a higher alcohol, preferably propanol, isopropanol or butanol, particularly preferably isopropanol, the nortropan-3-one compound, for example 8-benzylnortropan-3-one, can be isolated in the form of its hydrohalide of the formula XIV, preferably in the form of the hydrochloride of the formula XIVa, in very good yield and purity.

By virtue of the method according to the present technology, the production of nortropan-3-one compounds of the formula X, for example 8-benzylnortropan-3-one, or acid addition salts thereof, such as, for example, the hydrochloride, is possible under conditions which are advantageous from a processing and safety aspect and which are characterized in particular by a controlled gradual release of the gaseous carbon dioxide which forms as a result of the reaction. This is of great importance in particular for reactions on an industrial scale.

The process product is produced in high yield and in high purity, it being possible to easily separate off the formed by-products by simple methods and without loss of product of value. The nortropan-3-one compounds of the formula X obtained according to the present technology, in particular 8-benzylnortropan-3-one, and in particular the hydrohalide of the formula XIV released therefrom, preferably the hydrochloride of the formula XIVa, are thus suitable to a particular degree as starting material or intermediate for producing pharmaceutical active ingredients such as azoniaspironortropine ester halide compounds of the formula I, in particular trospium halides, on which particularly high requirements are placed with regard to purity and by-product spectrum.

### DEFINITIONS

The term “alkyl group” relates to a monovalent branched or unbranched saturated hydrocarbon chain having preferably 1 to 20 carbon atoms, preferably 1 to 10 carbon atoms, more preferably 1, 2, 3, 4, 5 or 6 carbon atoms. Examples of this term are groups such as methyl, ethyl, n-propyl, isopropyl, n-butyl, isobutyl, tert-butyl, n-hexyl, n-decyl, tetradecyl groups and the like.

The term “alkylene group” relates to a divalent radical of a branched or unbranched saturated hydrocarbon chain having preferably 1 to 20 carbon atoms, preferably 1 to 10 carbon atoms, more preferably 1, 2, 3, 4, 5 or 6 carbon atoms. Examples of this term are groups such as methylene group (—CH₂—), ethylene group (—CH₂CH₂—), the propylene isomers (such as —CH₂CH₂CH₂— and —CH(CH₃)CH₂—) and the like.

The term “alkenyl group” relates to a monovalent radical of a branched or unbranched unsaturated hydrocarbon group having preferably 2 to 20 carbon atoms, more preferably 2 to 10 carbon atoms and even more preferably 2, 3, 4, 5 or 6 carbon atoms and having 1 to 6, preferably one, double bond. Preferred alkenyl groups include ethenyl or vinyl (—CH═CH₂), 1-propylene or allyl (—CH₂CH═CH₂), isopropylene (—C(CH₃)═CH₂), bicyclo[2.2.1]heptene groups and the like. If an alkenyl group is bonded to a nitrogen atom, the double bond may not be located in the alpha position relative to the nitrogen atom.

The term “alkenylene group” relates to a divalent radical of a branched or unbranched unsaturated hydrocarbon group having preferably 2 to 20 carbon atoms, more preferably 2 to 10 carbon atoms and even more preferably 2, 3, 4, 5 or 6 carbon atoms and having 1 to 6, preferably one, double bond.

The term “cycloalkyl group” relates to a cyclic alkyl group having 3 to 20 carbon atoms and one single cyclic ring or two or more condensed rings. Such cycloalkyl groups include, for example, individual ring structures, such as cyclopropyl, cyclobutyl, cyclopentyl, cyclohexyl, cycloheptyl, cyclooctyl groups and the like or multiring structures such as adamantanyl and bicyclo[2.2.1]heptane groups or a cyclic alkyl group to which an aryl group is condensed, such as an indane group and the like.

The term “heterocyclyl group” relates to a saturated or partially unsaturated group with one single ring or two or more condensed rings which has in the ring 1 to 40 carbon atoms and 1 to 10 heteroatoms, preferably 1 to 4 heteroatoms, which are selected from nitrogen, sulfur, phosphorus and/or oxygen atoms.

The term “aryl group” relates to an aromatic carbocyclic group having 6 to 20 carbon atoms and a single ring (such as a phenyl group) or two or more rings (such as a biphenyl group) or two or more condensed (annelated) rings (such as naphthyl, anthryl, tetrahydronaphthyl, indane groups and the like). Preferred aryl groups include phenyl and naphthyl groups.

The term “heteroaryl group” relates to an aromatic group (i.e. an unsaturated group) which comprises 1 to 15 carbon atoms and 1 to 4 heteroatoms which are selected from oxygen, nitrogen and sulfur atom, within at least one ring.

The term “acyl group” refers to the group —C(O)R, in which R is selected from the group consisting of hydrogen atom, optionally substituted alkyl group, optionally substituted cycloalkyl group, optionally substituted heterocyclyl group, optionally substituted aryl group and optionally substituted heteroaryl group.

The term “carboxyl ester group” relates to the group —C(O)OR, in which R is selected from the group consisting of optionally substituted alkyl group, optionally substituted cycloalkyl group, optionally substituted heterocyclyl group, optionally substituted aryl group and optionally substituted heteroaryl group.

The term “halogen atom” or “halogen” relates to fluorine, chlorine, bromine and iodine atoms.

The term “hydrocarbon radical” relates to a monovalent group of carbon and hydrogen atoms. Hydrocarbon radicals may be straight-chain or branched, contain ring structures and be saturated or unsaturated. Hydrocarbon radicals preferably have 1 to 20 carbon atoms, preferably 1 to 10 carbon atoms, more preferably 1, 2, 3, 4, 5 or 6 carbon atoms. Examples of hydrocarbon radicals are alkyl, alkenyl, alkynyl, cycloalkyl and aryl groups, and combinations of a hydrocarbon bridge and an alkyl, alkenyl, alkynyl, cycloalkyl or aryl group.

The term “hydrocarbon bridge” relates to a divalent group of carbon and hydrogen atoms. Hydrocarbon bridges may be straight-chain or branched, contain ring structures and be saturated or unsaturated. Hydrocarbon bridges preferably have 1 to 20 carbon atoms, preferably 1 to 10 carbon atoms, more preferably 1, 2, 3, 4, 5 or 6 carbon atoms. Examples of hydrocarbon bridges are alkylene, alkenylene, alkynylene, divalent cycloalkyl and divalent aryl groups and combinations thereof.

“Optionally” means that the event described subsequently or the state described subsequently may or may not arise and that the description includes cases where the event or the state does arise and includes cases where this is not the case.

The term “substituted” in connection with a compound, a group or a radical means that in the compound, the group or the radical one or more hydrogen atoms are in each case replaced by a substituent. Preferably, a substituted compound, a substituted group or a substituted radical has 1, 2, 3, 4 or 5 substituents, more preferably 1, 2 or 3 substituents. These substituents are preferably independently of one another selected from the group consisting of alkyl, alkenyl, alkynyl, alkoxy, cycloalkyl, cycloalkenyl, acyl, acylamino, acyloxy, amino, aminocarbonyl, alkoxycarbonylamino, azide, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl groups. Unless limited by the definition, all substituents may be optionally further substituted by 1, 2 or 3 substituents which are selected from alkyl, carboxy, carboxyalkyl, aminocarbonyl, hydroxy, alkoxy, halogen, CF₃, amino, substituted amino, cyano and —S(O)ₙR groups, in which R is an alkyl, aryl or heteroaryl group and n has the value 0, 1 or 2. Preferably, a substituted compound, a substituted group or a substituted radical has 1, 2 or 3 substituents selected from the group consisting of alkyl, alkenyl, cycloalkyl and aryl group.

The present technology is described in detail by the examples below, which are used only for illustrative purposes and are not meant to be limiting. Owing to the description and the examples, further embodiments which are likewise included in the present technology are accessible to the skilled worker.

### EXAMPLES

The examples below serve to illustrate the present technology without limiting it in any way:

### Example 1

1,4-Dichlorobutane (52.4 g, 0.4 mol) and DMF (100 ml) were initially introduced in a reaction vessel at 80° C. and a solution of endo-nortropine (25.5 g, 0.2 mol), and DBU (88.8 g, 0.3 mol) in N,N-dimethylformamide (DMF, 150 ml) was added over the course of 1 h and the reaction mixture was afterstirred for a further 1 h at 80° C.

1,1′-Carbonyldiimidazole (48.6 g) and DMF (100 ml) were initially introduced in a second reaction vessel and a solution of benzylic acid (69.2 g, 0.3 mol) in DMF (150 ml) was added over the course of 30 min at a temperature of 20° C. The reaction mixture was stirred for 1 h at 20° C.

At 80° C., N,N-dimethylaminopyridine (2.5 g, 0.02 mol) and the suspension of benzylic acid imidazolide prepared as described above were added to the reaction mixture of 1,4-dichlorobutane and endo-nortropine over the course of 5 min and the resulting mixture was further stirred for 1 h at 80° C.

The reaction mixture was cooled to 0° C., filtered and the yellow filter residue was washed twice with in each case 50 ml of DMF and twice with in each case 100 ml of acetone. The filter cake was dried with nitrogen and 82.6 g of crude trospium chloride were obtained in the form of a colorless solid.

77 g of the crude product were dissolved in 535 ml of n-propanol and 5 ml of a 5-6 molar solution of HCl in isopropanol at a temperature of 90° C. After filtration, the solution was cooled to 0° C. The filter cake was washed again twice with in each case 100 ml of acetone and dried at 50° C. and 30 mbar for 12 h. This gave trospium chloride (61.2 g, 78%) in the form of a colorless solid.

### Example 2

1,4-Dichlorobutane (5.24 g, 40 mmol) and DMF (10 ml) were heated to 80° C. under nitrogen and endo-nortropine (2.55 g, 20 mmol) and DBU (4.57 g, 30 mmol) dissolved in DMF (15 ml) were added dropwise. After one hour, the mixture was cooled to 20° C., benzylic acid (6.92 g, 30 mmol) and CDI (4.86 g, 30 mmol) were added, the mixture was stirred for one hour, DMAP (250 mg, 2 mmol) was added and the mixture was heated at 80° C. for one hour. Upon cooling to 0° C., the product of value crystallized out and was filtered off, washed with DMF (2×5 ml) and acetone (2×10 ml) and dried in a stream of nitrogen. Trospium chloride, crude (7.1 g, 82.2% strength, 68%) was obtained in the form of a virtually colorless, crystalline solid.

### Example 3

Preparation of solution 1: 365.3 g of 1,3-acetone-dicarboxylic acid were dissolved in 1050 g of 5 molar sodium hydroxide solution while cooling in an ice bath.

Preparation of solution 2: 626.5 g of 32% strength hydrochloric acid and 187.5 g of benzylamine were dissolved in 1150 ml of completely demineralized water. 363.8 g of 2,5-dimethoxytetrahydrofuran were added to this solution. This mixture was stirred at room temperature for half an hour.

Solution 2 and solution 1 were added in parallel over the course of three hours to a stirred reactor with a volume of 4 l. Here, solution 2 was introduced into the reactor continuously and solution 2 was metered in such that the pH of the resulting reaction mixture was 3.5+/−0.1 over the entire duration of the addition. After an addition time of three hours, the remainder of solution 1 was added over the course of 10 minutes. The resulting reaction suspension was then stirred for two hours at room temperature.

During this addition, with a time delay of about 10 minutes, the continuous release of carbon dioxide was observed. Overall, in the course of the reaction, about 20 liters of carbon dioxide were released.

The solution was then rendered basic by adding sodium hydroxide solution to a pH of 10. To extract the product of value as free amine, 300 ml of saturated sodium chloride solution and 500 ml of ethyl acetate were added. After phase separation had taken place, the aqueous phase was afterextracted with 250 ml of ethyl acetate. The combined organic phases were admixed with 1000 ml of ethyl acetate, which were then removed again by azeotropic distillation under reduced pressure (35° C., 120 mbar).

The organic solution was then admixed with 650 ml of isopropanol and cooled to 0° C. To isolate the product, an acidic pH was then established by adding 330 g of 5 to 6 molar isopropanolic HCl solution. This gave 8-benzylnortropan-3-one hydrochloride in the form of a solid which was isolated by filtration. The resulting product had a content of 8-benzylnortropan-3-one hydrochloride of 75 by weight.

For further purification, the isolated solid was suspended, still solvent-moist, in 3570 g of methanol. Then, by means of filtration using a filter auxiliary, the solids fraction was removed and finally a solvent exchange of methanol to isopropanol was carried out and the 8-benzylnortropan-3-one hydrochloride was isolated in the form of a solid. This gave 302 g of 8-benzylnortropan-3-one hydrochloride with a content of 99% by weight, corresponding to a yield of 69%.

### Example 4

460 g of water, 250 g of 32% strength hydrochloric acid, 75.2 g of benzylamine and 145.2 g of 2,5-dimethoxytetrahydrofuran were initially introduced in a round-bottomed flask with a volume of 2 l. This mixture was stirred for 30 minutes at room temperature. Then, over the course of 45 minutes, 336 ml of a solution of 146 g of 1,3-acetonedicarboxylic acid and 420 g of a 5 molar sodium hydroxide solution were added, during which the pH increased to 1.

130 ml of a mixture of 146 g of 1,3-acetonedicarboxylic acid and 420 g of a 5 molar sodium hydroxide solution were then added, spread over 3 hours. During this, the pH increased to 4.8. During this increase in the pH, carbon dioxide was released. This mixture was then stirred for a further 2 hours at room temperature. Here, a pH of 6.1 was obtained. The solution was then rendered basic by adding sodium hydroxide solution to a pH of 10. To extract the product of value as free amine, 120 ml of saturated sodium chloride solution and 200 ml of ethyl acetate were added.

After phase separation had taken place, the aqueous phase was afterextracted twice with 100 ml of ethyl acetate. Then, under atmospheric pressure, the ethyl acetate was removed and replaced with methanol. Then, by adding 80 g of 32% strength hydrochloric acid, a pH of less than 3 was established. Afterstirring was then carried out for a further 20 minutes at room temperature and the impurity was removed by filtration. The filtrate was diluted with a further 980 ml of methanol and treated with 100 g of activated carbon. Following renewed filtration, the solvent methanol was again replaced by isopropanol and the resulting solid was isolated by filtration.

Finally, the latter was washed again twice with isopropanol. This gave 126.2 g of 8-benzylnortropan-3-one hydrochloride with a content of 96% by weight, corresponding to a yield of 72%.

1. A method for producing azoniaspironortropine ester halide compounds of the formula I 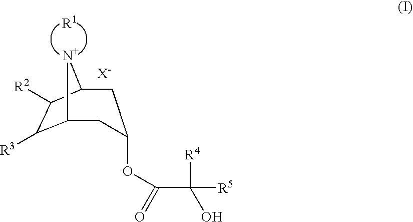
where
X is a halogen atom,
R¹ is selected from the group consisting of optionally substituted alkylene group, optionally substituted alkenylene group and the group —R—Z—R—, where each R, independently of the others, is a covalent bond, an optionally substituted alkylene group or an optionally substituted alkenylene group, where both R groups may not be a covalent bond at the same time, and Z is an optionally substituted cycloalkyl group, an optionally substituted heterocyclyl group, an optionally substituted aryl group or an optionally substituted heteroaryl group, R² and R³, independently of one another, are selected from the group consisting of hydrogen, optionally substituted alkyl group and optionally substituted cycloalkyl group, and
R⁴ and R⁵, independently of one another, are selected from the group consisting of hydrogen, halogen atom, optionally substituted alkyl group, optionally substituted alkenyl group, optionally substituted cycloalkyl group, optionally substituted heterocyclyl group, optionally substituted aryl group and optionally substituted heteroaryl group,
by reacting a compound of the formula II 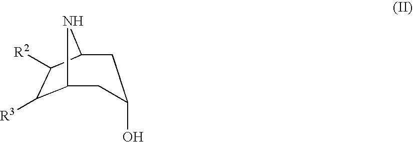
where
R² and R³ each have the same meaning as in formula I,
with a compound of the formula III
X—R¹—X¹ (III)
where
X and R¹ each have the same meaning as in formula I, and
X¹ is a halogen atom,
and with a compound of the formula IV 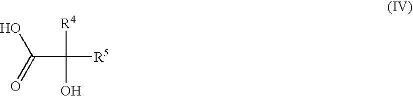
where
R⁴ and R⁵ each have the same meaning as in formula I,
in the presence of a base of the formula V 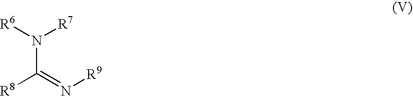
where
R⁶, R⁷ and R⁹ may be identical or different and, independently of one another, are hydrogen or an optionally substituted, saturated or unsaturated hydrocarbon radical, where R⁷ and R⁹ may together also form an optionally substituted, saturated or unsaturated hydrocarbon bridge, and
R⁸ is hydrogen or an optionally substituted, saturated or unsaturated hydrocarbon radical or the radical —NR¹⁰R¹¹, where the radicals R¹⁰ and R¹¹ may be identical or different and are hydrogen or an optionally substituted, saturated or unsaturated hydrocarbon radical, where the radicals R⁶ and R⁸ may together also form an optionally substituted hydrocarbon bridge,
and in the presence of at least one activating reagent selected from the group of activating reagents 1,1′-carbonyldiimidazole, 1,1′-thiocarbonyldiimidazole and thionyldiimidazole.

2. The method as claimed in claim 1, characterized in that R¹ is a 1,4-butylene group.

3. The method as claimed in claim 1, characterized in that R² and R³ are hydrogen.

4. The method as claimed in claim 1, characterized in that R⁴ and R⁵ are in each case a phenyl group.

5. The method as claimed in claim 1, characterized in that X and X¹ are in each case a chlorine atom.

6. The method as claimed in claim 1 for producing trospium halide of the formula Ia 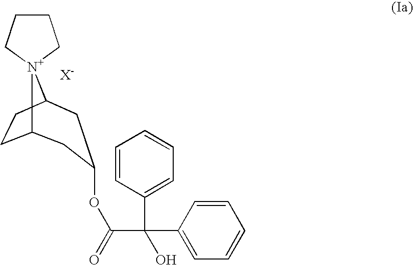
where
X is chlorine, bromine or iodine,
by reacting endo-nortropine of the formula IIa 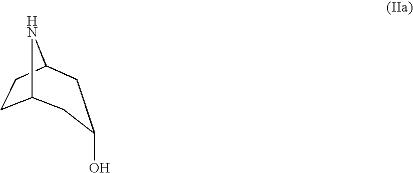
with 1,4-dihalobutane of the formula IIIa 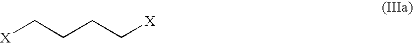
where
X in each case has the same meaning as in formula Ia
and with benzylic acid of the formula IVa 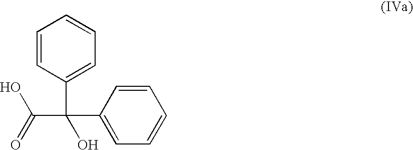
in the presence of a base of the formula Va 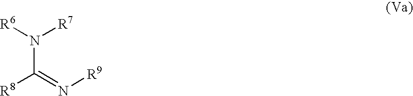
in which the radicals
R⁶, R⁷ and R⁹ may be identical or different and, independently of one another, are hydrogen or a saturated or unsaturated hydrocarbon radical having 1 to 4 carbon atoms, where R⁷ and R⁹ may together also form a saturated or unsaturated hydrocarbon bridge having 3 to 6 carbon atoms, and
R⁸ is hydrogen or a saturated or unsaturated hydrocarbon radical having 1 to 4 carbon atoms or the radical —NR¹⁰R¹¹, where the radicals R¹⁰ and R¹¹ may be identical or different and are hydrogen or a saturated or unsaturated hydrocarbon radical having 1 to 4 carbon atoms, and where the radicals R⁶ and R⁸ may together also form a hydrocarbon bridge having 3 to 6 carbon atoms,
and in the presence of at least one activating reagent selected from the group of activating reagents 1,1′-carbonyldiimidazole, 1,1′-thiocarbonyldiimidazole and thionyldiimidazole.

7. The method as claimed in claim 1, characterized in that imidazole or 1,8-diazabicyclo[5.4.0]undec-7-ene is used as base of the formula V or Va.

8. The method as claimed in claim 1, characterized in that imidazole is used as base of the formula V or Va.

9. The method as claimed in claim 8, characterized in that the imidazole used is formed in situ as the reaction product of the activating reagent used with the compound of the formula IV or IVa.

10. The method as claimed in claim 1, characterized in that the reaction is carried out in N,N-dimethylformamide, N,N-dimethylacetamide or N-methylpyrrolidone or mixtures thereof as solvent.

11. The method as claimed in claim 1, characterized in that the compound of the formula II or IIa is present in the reaction mixture in a concentration in the range from 5 to 30% by weight.

12. The method as claimed in claim 1, characterized in that the method is carried out in the form of a single-stage method and no intermediates are isolated during it.

13. The method as claimed in claim 1, characterized in that the reaction is carried out in the presence of 4-(N,N-dimethylamino)pyridine as catalyst.

14. The method as claimed in claim 1, characterized in that the compound of the formula I or Ia obtained is treated with a solvent in which the compound of the formula I or Ia precipitates out and the hydrohalide of the base of the formula V or Va used remains dissolved.

15. The method as claimed in claim 14, characterized in that N,N-dimethylformamide is used as solvent.

16. A method for producing nortropan-3-one compounds of the formula X 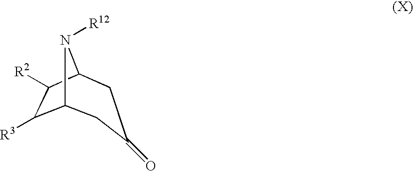
where
R² and R³, independently of one another, are selected from the group consisting of hydrogen, optionally substituted alkyl group and optionally substituted cycloalkyl group, and
R¹² is selected from the group consisting of optionally substituted alkyl group, acyl group and carboxyl ester group,
by reacting a compound of the formula XI
H₂N—R¹² (XI)
where
R¹² has the same meaning as in formula X,
with a compound of the formula XII 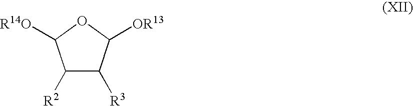
where
R² and R³ each have the same meaning as in formula X, and
R¹³ and R¹⁴, independently of one another, are selected from the group consisting of hydrogen, optionally substituted alkyl group, optionally substituted alkenyl group, optionally substituted cycloalkyl group and optionally substituted aryl group,
and 1,3-acetonedicarboxylic acid of the formula XIII 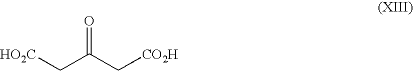
characterized in that 1,3-acetonedicarboxylic acid of the formula XIII is used in the form of an aqueous solution with a pH of greater than 7.

17. The method as claimed in claim 16, characterized in that R² and R³ are hydrogen.

18. The method as claimed in claim 16, characterized in that R¹² is a benzyl group.

19. The method as claimed in claim 16 for producing 8-benzylnortropan-3-one of the formula Xa 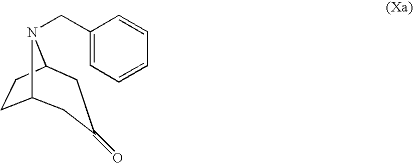
by reacting benzylamine of the formula XIa 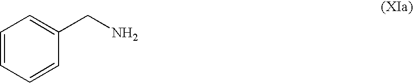
with a compound of the formula XIIa 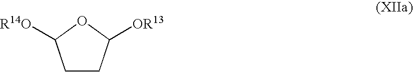
where the radicals
R¹³ and R¹⁴ are identical or different and are in each case hydrogen or an unbranched or branched C₁- to C₆-alkyl group,
and 1,3-acetonedicarboxylic acid of the formula XIII 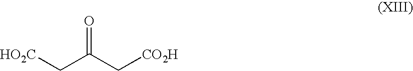
characterized in that 1,3-acetonedicarboxylic acid of the formula XIII is used in the form of an aqueous solution with a pH of greater than 7.

20. The method as claimed in claim 16, characterized in that R¹³ and R¹⁴ are in each case a methyl group.

21. The method as claimed in claim 16, characterized in that the compound of the formula XI or XIa and the compound of the formula XII or XIIa are used in the form of an aqueous solution with a pH of less than 7.

22. The method as claimed in claim 21, characterized in that the aqueous solution with a pH of less than 7 comprises hydrochloric acid.

23. The method as claimed in claim 21, characterized in that during the reaction the compound of the formula XI or XIa and the compound of the formula XII or XIIa are initially introduced in the form of an aqueous solution with a pH of less than 7 in a reaction vessel, and 1,3-acetonedicarboxylic acid of the formula XIII is metered in to give a reaction mixture.

24. The method as claimed in claim 21 or 22, characterized in that during the reaction the compound of the formula XI or XIa and the compound of the formula XII or XIIa in the form of an aqueous solution with a pH of less than 7 and 1,3-acetonedicarboxylic acid of the formula XIII are simultaneously metered into a reaction vessel to give a reaction mixture.

25. The method as claimed in claim 23, characterized in that the reaction mixture has a pH of from 3 to 4.

26. The method as claimed in claim 16, characterized in that 1,3-acetonedicarboxylic acid of the formula XIII is used in the form of a solution in aqueous sodium hydroxide.

27. A method for producing nortropan-3-one hydrohalide compounds of the formula XIV 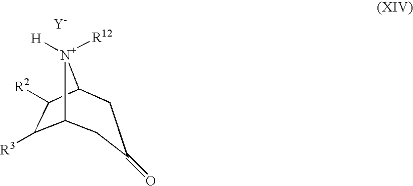
where
R² and R³, independently of one another, are selected from the group consisting of hydrogen, optionally substituted alkyl group and optionally substituted cycloalkyl group,
R¹² is selected from the group consisting of optionally substituted alkyl group, acyl group and carboxyl ester group, and
Y is a halogen atom,
by producing a nortropan-3-one compound according to any one of claims 16 to 26 and then treating the nortropan-3-one compound produced in this way with a hydrohalic acid.

28. The method as claimed in claim 27, characterized in that the resulting nortropan-3-one hydrohalide compound of the formula XIV is suspended in methanol and purified by stirring.

29. The method as claimed in claim 27, characterized in that Y is a chlorine atom and the hydrohalic acid is hydrochloric acid.

30. The method as claimed in claim 27, characterized in that R² and R³ are hydrogen and R¹² is a benzyl group.

31. A method for producing endo-nortropine compounds of the formula II 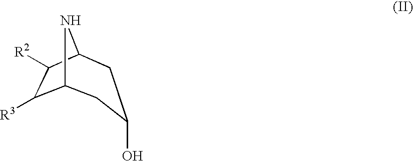
where
R² and R³, independently of one another, are selected from the group consisting of hydrogen, optionally substituted alkyl group and optionally substituted cycloalkyl group,
by producing a nortropan-3-one compound as claimed in any one of claims 16 to 26 or by producing a nortropan-3-one hydrohalide compound as claimed in any one of claims 27 to 30 and subsequently converting the nortropan-3-one compound or nortropan-3-one hydrohalide compound to an endo-nortropine compound of the formula II.

32. The method for producing azoniaspironortropine ester halide compounds as claimed in claim 1, additionally comprising the production of an endo-nortropine compound of the formula II 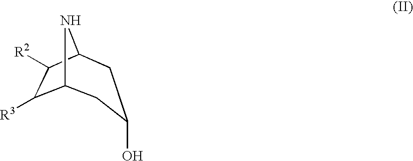
where
R² and R³, independently of one another, are selected from the group consisting of hydrogen, optionally substituted alkyl group and optionally substituted cycloalkyl group,
by producing a nortropan-3-one compound as claimed in any one of claims 16 to 26 or by producing a nortropan-3-one hydrohalide compound as claimed in any one of claims 27 to 30 and subsequently converting the nortropan-3-one compound or nortropan-3-one hydrohalide compound to an endo-nortropine compound of the formula II.
